(19) United States
(12) Patent Application Publication
(10) Pub. No.: US 2015/0347712 A1
(43) Pub. Date: Dec. 3, 2015

(54) SYSTEM AND METHOD FOR PRODUCT DISTRIBUTION AND TRACKING

(71) Applicant: Amerisourcebergen Specialty Group, Inc., Frisco, TX (US)

(72) Inventors: Chris Flori, Dallas, TX (US); Dustin Roller, Frisco, TX (US)

(73) Assignee: Amerisourcebergen Specialty Group, Inc., Frisco, TX (US)

(21) Appl. No.: 14/288,139

(22) Filed: May 27, 2014

(51) Int. Cl. G06F 19/00; B65D 75/36; A61J 1/03; B65D 65/42; G06K 7/10; G06Q 10/08

(52) U.S. Cl. CPC G06F 19/3456; G06K 7/10366; G06Q 10/087; A61J 1/035; B65D 65/42; B65D 75/367; A61J 2205/60; B65D 2565/382

(57) ABSTRACT

A method for distributing products includes placing each of a plurality of products into a separate cell of a package, the package having a plurality of cells. The method also includes covering the cells of the package with a cover layer having a plurality of radio frequency identification (RFID) tags, such that each RFID tag is aligned with a corresponding cell, and sealing the package. The method further includes associating information about each product with the corresponding RFID tag in one or more first digital records and storing the first digital records in a memory.

### TECHNICAL FIELD

The present disclosure is generally related to product distribution systems, and more particularly, to a system and method for product distribution and subsequent tracking of usage of the product.

### BACKGROUND

Product tracking is of importance to any manufacturing, distribution, or sales enterprise. It can be particularly important in the pharmaceutical area, where many products must be carefully identified and tracked from manufacture until administered to a patient. Typical known means of tracking pharmaceuticals involve manual record keeping and identifying products according to written labels. Even when some functions are automated, the ability to track the products stops once the products are delivered to the patient, who may not be diligent in accurately using the product or reporting its usage to a caregiver.

### SUMMARY

According to one embodiment, a method for distributing products includes placing each of a plurality of products into a separate cell of a package, the package having a plurality of cells. The method also includes covering the cells of the package with a cover layer having a plurality of radio frequency identification (RFID) tags, such that each RFID tag is aligned with a corresponding cell, and sealing the package. The method further includes associating information about each product with the corresponding RFID tag in one or more first digital records and storing the first digital records in a memory.

According to another embodiment, a method for tracking product usage includes establishing communication with a radio frequency identification (RFID) enabled package, the package having a plurality of cells, each cell containing a product, each cell having a corresponding RFID tag on a surface of the cell. The method also includes determining first information associated with the products in the package by wirelessly monitoring each RFID tag. The method further includes detecting, after removal of the package from a reader, that the package has been returned to the reader. The method still further includes determining, after the detection that the package has been returned to the reader, second information associated with the products in the package by wirelessly monitoring each RFID tag again, wherein at least one of the RFID tags is broken and unreadable. The method also includes transmitting the first and second information to a network node associated with at least one vested entity associated with the products. The first information is different than the second information due to the at least one broken RFID tag.

According to yet another embodiment, an apparatus for tracking product usage includes a radio frequency identification (RFID) reader, a data processing system, and a network interface. The RFID reader is configured to establish communication with a RFID enabled package, the package having a plurality of cells, each cell containing a product, each cell having a corresponding RFID tag on a surface of the cell. The data processing system is configured to determine first information associated with the products in the package by wirelessly monitoring each RFID tag; determine, after removal of the package from the apparatus, that the package has been returned to the apparatus; and determine, after the package has been returned to the reader, second information associated with the products in the package by wirelessly monitoring each RFID tag again, wherein at least one of the RFID tags is broken and unreadable. The network interface is configured to transmit the first and second information to a network node associated with at least one vested entity associated with the products. The first information is different than the second information due to the at least one broken RFID tag.

Before undertaking the DETAILED DESCRIPTION OF THE INVENTION below, it may be advantageous to set forth definitions of certain words or phrases used throughout this patent document: the terms “include” and “comprise,” as well as derivatives thereof, mean inclusion without limitation; the term “or” is inclusive, meaning and/or; the phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like; and the term “controller” means any device, system or part thereof that controls at least one operation, whether such a device is implemented in hardware, firmware, software or some combination of at least two of the same. It should be noted that the functionality associated with any particular controller may be centralized or distributed, whether locally or remotely. Definitions for certain words and phrases are provided throughout this patent document, and those of ordinary skill in the art will understand that such definitions apply in many, if not most, instances to prior as well as future uses of such defined words and phrases.

### BRIEF DESCRIPTION OF THE DRAWINGS

For a more complete understanding of the present disclosure, and the advantages thereof, reference is now made to the following descriptions taken in conjunction with the accompanying drawings, wherein like numbers designate like objects, and in which:

FIG. 1 illustrates an example network topology of a product tracking system according to this disclosure;

FIG. 2 illustrates an example smart package reader according to this disclosure;

FIG. 3 illustrates an example smart package according to this disclosure;

FIG. 4 illustrates an example process for assembling a smart package or blister pack according to this disclosure;

FIG. 5 illustrates an example method for distributing products in a smart package, according to this disclosure; and

FIG. 6 illustrates an example method for tracking usage of products in a smart package, according to this disclosure.

### DETAILED DESCRIPTION

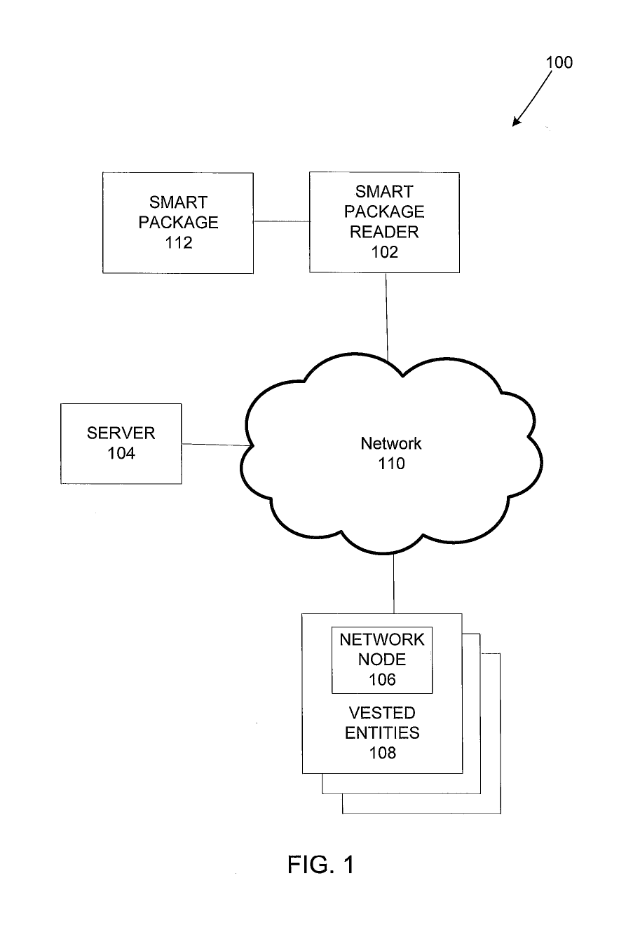

FIGS. 1 through 6, discussed below, and the various embodiments used to describe the principles of the present disclosure in this patent document are by way of illustration only and should not be construed in any way to limit the scope of the disclosure. Those skilled in the art will understand that the principles of the present disclosure may be implemented in any suitably arranged device. The numerous innovative teachings of the present disclosure will be described with particular reference to the presented embodiments.

Embodiments of the present disclosure include a system and method for packaging, distributing, and tracking products, such as pharmaceutical products, and sharing information associated with the products with one or more entities, such as healthcare professionals, vested in the use of the products. Various embodiments use a radio frequency identifier (RFID) enabled blister pack and a product tracking system that reads information from the RFID enabled blister pack and tracks or otherwise maintains an ongoing status of one or more products associated with the RFID enabled blister pack. In contrast to other packages that include a single RFID tag that is associated with the entire package, the disclosed RFID enabled blister pack includes a unique RFID tag for each cell of the package.

FIG. 1 illustrates an example network topology of a product tracking system 100 according to this disclosure. The embodiment of the product tracking system 100 illustrated in FIG. 1 is for illustration only. Other embodiments could be used without departing from the scope of this disclosure.

The product tracking system 100 includes one or more smart package readers 102 each coupled to at least one smart package 112, a server 104, and one or more network nodes 106 of one or more corresponding vested entities 108 that communicate through a network 110, such as the Internet.

The smart package 112 stores an inventory of one or more products. In certain embodiments, the smart package 112 is a RFID enabled blister pack that includes a unique RFID tag for each cell in the blister pack. In certain embodiments, the smart package reader 102 includes a RFID tag reader capable of reading information from each RFID tag associated with the smart package 112. Information about the products in the smart package 112 can be read by the corresponding smart package reader 102. As will be described in detail below, the server 104 periodically receives product information of the smart package 112 from the smart package reader 102 through the network 110. The server 104 stores the product information, and transmits the product information to network nodes 106 associated with one or more vested entities 108 associated with the products.

Products stored in the smart package reader 102 may be any suitable type for which an ongoing, relatively up-to-date status may be maintained and shared with any entities vested in the product. In certain embodiments, the product may be a pharmaceutical product that treats an ailment of a patient. In particular embodiments, the product must be carefully tracked to ensure proper usage by a patient, or for financial purposes. In other embodiments, the product may include non-pharmaceutical products.

The vested entities 108 may include those entities having a vested interest in the use and inventory of the product. For products such as pharmaceutical products, these vested entities may include a healthcare professional who administers the pharmaceutical (e.g., a doctor or nurse), an office administrative professional responsible for inventory management (e.g., an office manager, accounting professional, or bookkeeper), a pharmacy that provides the pharmaceutical product to the patient, a payer of the pharmaceutical product (e.g., an insurance provider), a manufacturer or distributor of the pharmaceutical product, or any other suitable entity.

Each vested entity 108 may receive up-to-date product information via its associated network node 106. The network nodes 106 may be any computing system having one or more processors that execute instructions stored in a memory. Examples of such a computing system include personal computers, mainframe computers, laptop computers, personal digital assistants, cellular telephones, smart phones, and the like. Each network node 106 may include any type of application or portal (e.g., a web portal, a local computer application, or a mobile application or “app”) to access and interact with the product information from one or more smart package readers 102.

In one embodiment, the network node 106 used by a vested entity 108 may periodically receive status information of products within the smart package 112 from the smart package reader 102 via the server 104. In another embodiment, the network node 106 transmits the product information to at least one vested entity 108 associated with the products in response to a request for the status information from the vested entity 108.

Access to the product information may be protected from illicit access via an authentication process. For example, the server 104 may use a password protected login session to restrict access to only registered vested entities 108. As another example, the server 104 may use a public-private key authentication architecture for automated, periodic access to information by network nodes used by each vested entity 108.

In certain embodiments, the server 104 may maintain an account for each vested entity such that the type and level of product information may be allocated independently for each vested entity 108. For an example in which the product is a pharmaceutical product, an office manager may wish to maintain an accurate status of the amount of product used for ordering and financial purposes, while a healthcare professional may wish to know which patients received and used the product, and in what amounts, for patient care. Thus, the account associated with the office manager may be allocated to receive only product inventory information for ordering and billing, while the healthcare professional may be allocated to receive product use information as well as related patient information associated with the use of the pharmaceutical product.

In certain embodiments, information transmitted through the network 110 may be encrypted to maintain its integrity and/or to thwart its illicit use. For example, the encryption of information may reduce the possibility of its modification by sniffing packets transmitted by the smart package reader 102 and spoofing these packets with illicit data. As another example, the information may be encrypted for compliance with certain governmental privacy requirements, such as the Health Insurance Portability and Accountability Act (HIPAA), which requires that only certain authorized entities have access to medical records of the patients associated with the smart package 112.

The network 110 can be implemented using any known networking technology, such as a public or private network or as direct communications, and is may be implemented using the Internet to communicate between each system. The network 110 can be implemented using multiple technologies, and can be implemented using multiple separate networks.

The product tracking system 100 as shown and described may include a single smart package reader 102 or may include multiple smart package readers 102. Likewise, each smart package reader 102 may be capable of reading product status information from one smart package 112 or from multiple smart packages 112. The server 104 may perform overall product tracking functions for the multiple smart package readers 102. In general, the server 104 communicates on a regular basis with each smart package reader 102 to monitor status and usage of the products in each smart package 112. The server 104 can also monitor other status information of each smart package reader 102 according to one or more sensor devices configured in the smart package reader 102. The server 104 includes a database that maintains a current inventory of the product, the product inventory assigned to each smart package reader 102, and other information regarding each smart package reader 102.

The server 104 may also track all products from time of purchase and receiving into a warehouse, to placement in the smart package 112, to storage in the smart package reader 102, to removal from the smart package 112. The server 104 may periodically receive communications from each smart package reader 102 including the current product list, the consumed product list, and other information, such as product serial numbers, lot numbers, and patient information. These communications can be initiated by the server 104, by polling of the smart package reader 102, or can be initiated by the smart package reader 102. The server 104 may be further configured to send control instructions to the smart package reader 102. The server 104 may be further configured to, in some embodiments, analyze product consumption data according to status data of each smart package 112 received from the smart package reader 102, as described below. For ease of reference, the term “serial number” will be used herein to refer to a unique identifier, although those of skill in the art will recognize that any suitable style of unique identifier can be used.

The server 104 may include a web server interface to allow management using a standard web browser interface. At least some data sent and received by the server 104 may be in extensible markup language (XML) format. The server 104 maintains at least one database for product inventory data, which in a particular embodiment, is a structured query language (SQL) database.

Although FIG. 1 illustrates one example product tracking system 100 for managing and sharing product information with one or more vested entities 108, various changes may be made to FIG. 1. For example, in some embodiments, product information may be stored in a memory configured in the smart package reader 102 and periodically downloaded to a portable memory device, such as a flash memory card, which is then physically transported to, and accessed by a network node of a vested entity such that the communication network may not be necessary. In other embodiments, for security reasons, the product information may not be stored in the smart package reader 102. In yet other embodiments, more than one of the smart package reader 102, the server 104, and the network nodes 106 administered by the vested entities 108 may be combined into a single computing system in which communication of product information may be provided by internal system calls between each of its users.

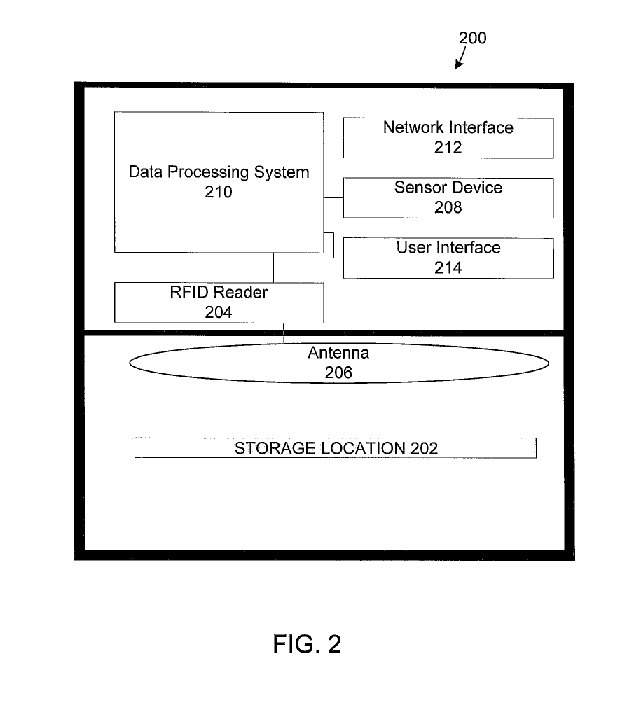

FIG. 2 illustrates an example smart package reader 200 according to this disclosure. In some embodiments, the smart package reader 200 may represent the smart package reader 102 as described above with reference to FIG. 1. The embodiment of the smart package reader 200 illustrated in FIG. 2 is for illustration only. Other embodiments could be used without departing from the scope of this disclosure.

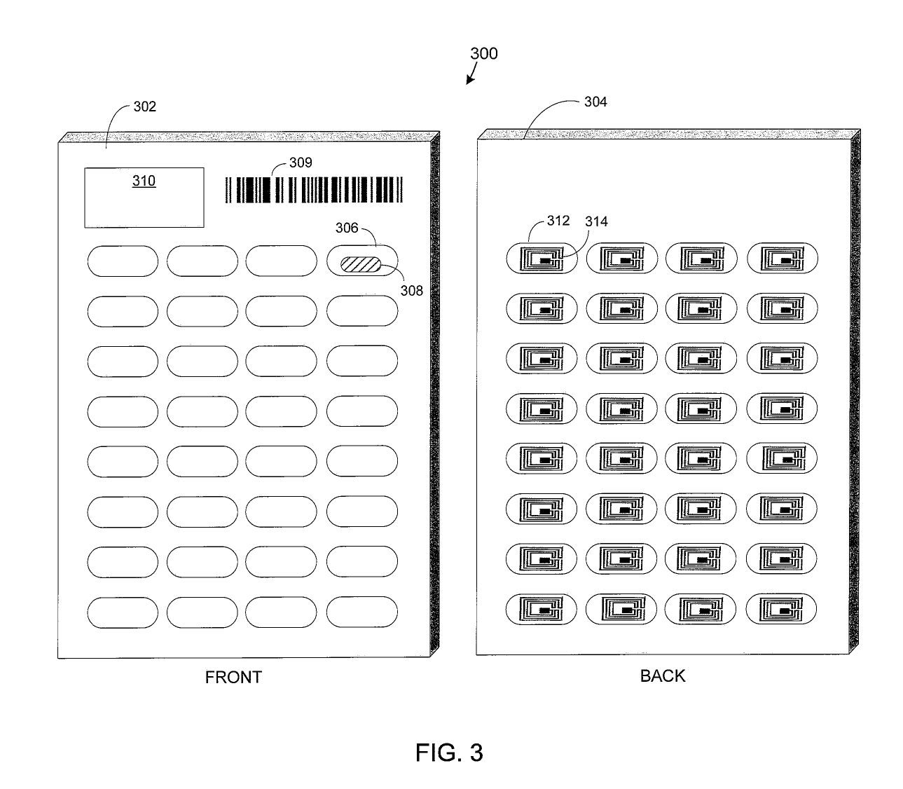

The smart package reader 200 includes a designated storage location 202 for storage of one or more smart packages 300 (FIG. 3). In some embodiments, the storage location 202 may be one or more slots into which a single smart package 300 (in the form of a blister card) may be inserted. In other embodiments, the storage location 202 may be a flat surface onto which a smart package 300 is placed. In still other embodiments, the storage location 202 may be a closable cabinet into which one or more smart packages 300 may be placed. The storage location 202 may also represent any other suitable location associated with storing one or more smart packages 300. In embodiments where the storage location 202 includes a cabinet, the smart package reader 200 may include a refrigeration and/or heating system for maintaining a temperature of the storage location 202 at any desired temperature. For refrigerated use, a conventional refrigeration unit can be modified as described herein. For ambient-temperature use, a non-refrigerated cabinet can be used, or the refrigeration unit can be turned off or disconnected.

The smart package reader 200 also includes a RFID reader 204 to wirelessly and automatically detect and identify the contents of the smart package 300 associated with the storage location 202. The smart package reader 200 includes one or more RFID antennas 206 coupled to the RFID reader 204 that are capable of scanning the contents of the smart package 300. The smart package reader 200 may also include one or more optional sensor devices 208, such as a thermometer, a door-open sensor, an occupied-slot sensor, a power sensor and optional backup power supply, a GPS locating device, or any other suitable sensor device.

The smart package reader 200 also includes a data processing system 210 that communicates with and controls the RFID reader 204. The data processing system 210 also includes communications software for communicating as described in detail below. The data processing system 210 also communicates with and controls the optional sensor devices 208 described above. The data processing system 210 may be implemented using any appropriate technology and components, configured to operate as described herein. The data processing system 210 generally includes one or more processors and one or more memory units for storing data as described herein.

The smart package reader 200 also includes a network interface 212 for communication with other devices, such as the server 104 and/or network nodes 106 of vested entities 108 described in FIG. 1. The network interface 212 may be implemented using a wired communication medium such as an Ethernet or a telephone modem, or wireless communication medium such as a Global System for Mobile (GSM) communications architecture, Wi-Fi network (e.g., IEEE 802.11), a cable modem system, or any combination of these. In a particular embodiment, the network interface 212 communicates using an Internet Protocol. The network interface 212 allows the cabinet data processing system 210 to communicate with the server 104, and optionally with other smart package readers 200 using a mesh networking topology, direct cabling, or other technologies. Communications between the data processing system 210 and the server 104 may be implemented using any suitable data communications technology, or any combination thereof. In embodiments where multiple smart package readers 200 communicate with each other, they can be configured to communicate with the server 104 as a single unit with combined product information.

The smart package reader 200 also may include a user interface 214 for interacting with a user of the smart package reader 200. The user interface 214 includes one or more user input devices and one or more displays, such as a customizable electronic display. In a particular embodiment, the one or more input devices and the one or more displays could be integrated into one unit, such as a touch screen. The user interface 214 is connected to the data processing system 210 and is configured to receive user input and display status or informational messages related to the status of the smart package reader 200 or the product information from the smart package 300, as described in greater detail below.

In use, the data processing system 210 performs periodic product scans, using the RFID reader 204, to uniquely identify each smart package 300 stored at the storage location 202 and read product information associated with each RFID tag of each smart package 300. If a new RFID tag is found during any scan, the data processing system 210 notes the RFID tag (and associated product) and stores it to a current product list for that smart package 300. Similarly, if a specific RFID tag is no longer detected during a periodic scan, because the smart package 300 has been removed or the RFID tag has been damaged, the data processing system 210 notes the missing RFID tag (and associated product) and removes it from the current product list for the smart package 300. The identifiers of such removed products may also be stored in a “consumed product” list in the data processing system 210. Timestamp information may be recorded and included with the data records to indicate when a particular product was added or removed.

In this manner, the smart package reader 200 is configured to monitor the products associated with each smart package 300 by wirelessly detecting the RFID tags. The smart package reader 200 performs a periodic wireless scan to determine the current products in the inventory, and determines that a product has been removed from the inventory when the RFID tag corresponding to the product is not detected for a predetermined amount of time. In some embodiments, the current product list and consumed product list can be displayed on a display of the user interface 214.

Of course, the references herein to the inventory list and consumed product list are not intended to specify a data structure for this information, as this information can be stored in any number of forms within the scope of the disclosed embodiments. The term “list” is simply used for convenient reference.

In embodiments where the storage location 202 is a cabinet, the cabinet 202 may include a locking mechanism, or one or more individual locking compartments, to control access to the products. These locks can be any known technology, including key locks, digital keypad locks, biometric locks, etc. In some embodiments, the locking mechanism can be opened by entering a user authentication code via an input device of the user interface 214. The locking device can also be opened remotely if the data processing system 210 receives such a command from a particular node of the product tracking system 100.

The smart package reader 200 may include a low-power or power-failure detection device and a backup power supply. When a power failure or low-power condition is detected, the smart package reader 200 may generate an audible alarm, and can communicate with the server 104 to notify the existence of a problem.

FIG. 3 illustrates an example smart package 300 according to this disclosure. In some embodiments, the smart package 300 may represent the smart package reader 112 as described above with reference to FIG. 1. The embodiment of the smart package 300 illustrated in FIG. 3 is for illustration only. Other embodiments could be used without departing from the scope of this disclosure. The smart package 300 described herein is configured for storage of pharmaceutical products; however, the smart package 300 can be applicable for other products.

The smart package 300 is a “blister card” or “blister pack” that has a front side 302 and a back side 304. The front side 302 of the smart package 300 includes a plurality of cells 306 arranged in a number of rows and columns, where each cell 306 may contain a product 308. The front side 302 of the smart package 300 also includes a card identifier 309 and a card information block 310. The back side 304 of the smart package 300 includes a plurality of cell covers 312 aligned with the cells 306. Each cell cover 312 includes a RFID tag 314, where the RFID tag 314 includes identifying information capable of being read by the RFID reader 204.

The cells 306 of the smart package 300 are typically deformed, clear plastic cavities sized to accommodate a product 308, such as a pharmaceutical oral solid or capsule. The cells 306 are typically connected and formed by stamping or thermoforming a single sheet of plastic, such as polyvinyl chloride (PVC), as known as in the art. Of course, the cells may be formed of any other suitable material as well.

The card identifier 309 may be a bar code, QR code, magnetic strip, RFID tag, smart chip, or any other machine readable identifier that includes identifying information associated with the smart package 300 and the contents of the smart package 300 (i.e., the products 308). For example, in a case where the smart package 300 contains a prescription of medication, the card identifier 309 may include information that identifies a patient to whom the medication is prescribed, a caregiver that prescribed the medication, the type and dosage of the medication, the date of the prescription, a prescription serial number, or any other suitable information.

The information block 310 is an area in which human-readable information about the smart package 300 can be handwritten, printed, or typed. The information in the information block 310 may include some or all of the information identified by the machine-readable card identifier 308. That is, the information block 310 may include information that identifies a patient to whom the medication is prescribed, a caregiver that prescribed the medication, the type and dosage of the medication, the date of the prescription, a prescription serial number, or any other suitable information.

The cell covers 312 on the back side 304 of the smart package 300 include a thin layer or film that aligns with and covers an opening of each cell 306, thereby creating a sealed enclosure for the product 308 within the cell 306. The thin layer may be formed of paper, plastic, metallic foil, paraffin, any other suitable material, or any combination of these. In a particular embodiment, the thin layer may be a paraffin-coated paper. In some embodiments, the cell covers 312 may be individually mounted on a second layer that substantially covers the back side 304 of the smart package 300. In other embodiments, the cell covers 312 may be merely portions of a single layer that substantially covers the back side 304 of the blister card. The thin layer or layers that form each cell cover 312 are formed to be breakable when the product 308 (e.g., a tablet) inside the corresponding cell is pressed against the cell cover 312. That is, an individual can release the product 308 from a sealed cell 306 by pressing on the front side of the cell 306 and pushing the product 308 against the cell cover 312 and breaking the cell cover 312, as known in the art.

Each cell cover 312 includes a RFID tag 314 that is uniquely associated with, and physically aligned with, a corresponding cell 306. The RFID tag 314 is affixed to, or embedded within, one or more layers that form the cell cover 312. In some embodiments, each RFID tag 314 is adhered to one side of a paper film and then the paper film and the RFID tags are covered with a protective second film. Each RFID tag 314 includes one or more unique identifiers, such as a serial number, that can be used to link the product 308 in the corresponding cell 306 with the particular RFID tag 314. That is, information about each cell 306, the product 308, and the corresponding RFID tag 314 can be linked together in a one-to-one relationship. A RFID reader (e.g., the RFID reader 204) can read information from the RFID tag 314, and a data processing system (e.g., the data processing system 210) can determine corresponding information about the product 308 in the cell 306 associated with the RFID tag 314. Later, when the product 308 is removed from the cell 306 by pressing the product 308 through the corresponding cell cover 312, the corresponding RFID tag 314 is damaged and rendered unreadable. The RFID reader can then detect that the RFID tag 314 is unreadable, and the associated product 308 can be indicated as removed.

The smart package 300 provides a number of advantages over other RFID enabled packages. Some RFID enabled packages include a single RFID tag for an entire package. In such packages, the single RFID tag is connected to a web of contacts, where each contact is associated with a cell containing a product. Sophisticated circuitry, sometimes including a processor and even a power source, are required in these RFID enabled packages in order to be able to determine which cell is disturbed when a product is removed. In contrast, the smart package 300 features an individual passive RFID tag for each cell. The smart package 300 is inexpensive to produce, does not require an integrated power supply, and each RFID tag 314 is independently readable by many common RFID readers.

Although a “product” 308, as described herein, generally represents a single product, there can be two or more actual products, which are packaged together. For example, in the pharmaceutical context, a single dose, pill, or pre-filled syringe can comprise a single product, but multiple ones of these can be packaged together as a single product 308, depending on the requirements for using, dispensing, or billing for the product.

Although FIGS. 2 and 3 illustrate one example smart package reader 200 and smart package 300, various changes may be made to FIGS. 2 and 3. For example, each RFID tag 314 may wirelessly communicate with the RFID reader 204 of the smart package reader 200 using any suitable protocol. Additionally, the smart package 300 may include any suitable type other than a blister pack, such as a bottle or jar containing individually packaged products.

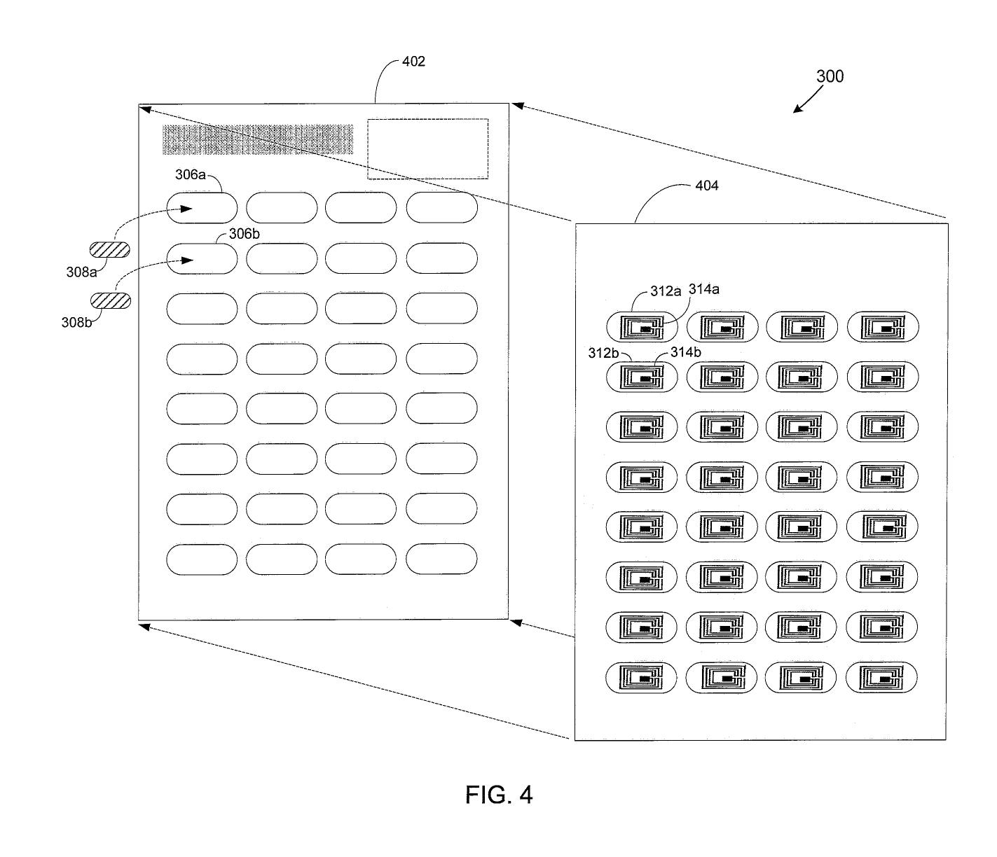

FIG. 4 illustrates an example process for assembling a smart package or blister pack 300 according to this disclosure. The embodiment of the smart package 300 illustrated in FIG. 4 is for illustration only. Other embodiments could be used without departing from the scope of this disclosure.

As shown in FIG. 4, a first layer 402 comprising the front side 302 of the smart package 300 is positioned face side down with openings to the cells 306 (represented by cells 306a, 306b) facing up. An operator (e.g., a pharmacist, a care provider, a dispensing or packaging machine, etc.) places a product 308 (represented by products 308a, 308b) into one, more than one, or all of the cells 306. For example, the product 308a is placed in the cell 306a, the product 308b is placed in the cell 306b, and so forth.

In some implementations, each product 308 is substantially identical. For example, for one pharmaceutical prescription, each product 308 may represent the same medication at the same dosage. In other implementations, certain products 308 may be different from other products 308. For example, in a prescription that uses a tapering schedule, the product 308a may be the same medication as the product 308b, but contain a different dosage. As another example, in a prescription that requires a combination of medications, the product 308a may be an entirely different medication than the product 308b. Depending on the prescription, one or more cells 306 may be left empty.

A second layer 404 that includes the cell covers 312 (represented by cell covers 312a, 312b) and the corresponding RFID tags 314 (represented by RFID tags 314a, 314b) is placed on the first layer 402. The second layer 404 is formed of paper, plastic, metallic foil, paraffin, any other suitable material, or any combination of these. In a particular embodiment, the second layer 404 may be a paraffin-coated paper. The cell covers 312 and RFID tags 314 are arranged on the second layer 404 in the same pattern as the cells 306 are arranged on the first layer 402. The second layer 404 is placed on the first layer 402 such that the cell cover 312a aligns with the cell 306a, the cell cover 312b aligns with the cell 306b, and so forth. In some embodiments, the second layer 404 is substantially the same size and shape as the first layer 402; thus, no trimming is necessary to fit the second layer 404 to the first layer 402. In other embodiments, the second layer 404 may be formed in a large sheet or roll such that trimming is required to fit the second layer 404 to the dimensions of the first layer 402.

The second layer 404 is then adhered to the first layer 402. In some embodiments, the first layer 402, the second layer 404, or both include a heat responsive glue, and once the second layer 404 is placed on the first layer 402, a short heating process heats the glue and seals the products 308 in the cells 306. In other embodiments, the first layer 402, the second layer 404, or both may include a “peel-and-stick” adhesive or another suitable adhesive to create the seal.

The sealed smart package 300 includes one or more products 308a, 308b in one or more cells 306a, 306b, where each cell is associated with a RFID tag 314a, 314b. At a predetermined stage in the assembling process, information about each product 308a, 308b is linked to an identifier associated with the corresponding RFID tag 314a, 314b. That is, the product 308a is linked to the RFID tag 314a, the product 308b is linked to the RFID tag 314b, and so on. Such information may be stored in a database or other memory associated with the server 104, a vested entity 108, the card identifier 309, or any other suitable location.

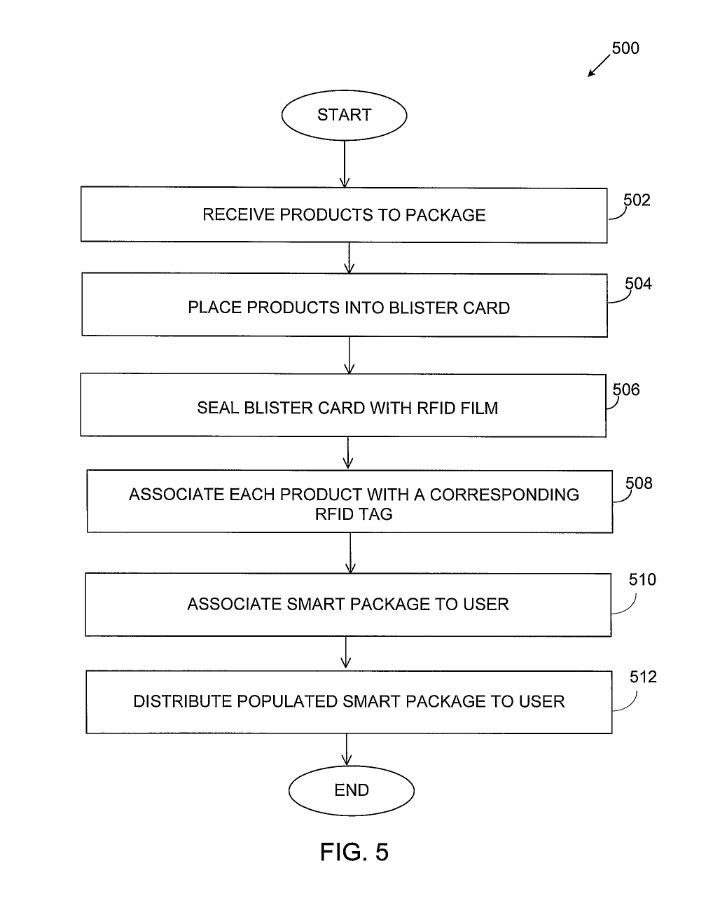

FIG. 5 illustrates an example method for distributing products in a smart package, according to this disclosure. In some embodiments, the method 500 may be performed in conjunction with the smart package 300 as described above with reference to FIGS. 3 and 4. The embodiment of the method 500 illustrated in FIG. 5 is for illustration only. Other embodiments could be used without departing from the scope of this disclosure. The method 500 is described in the context of pharmaceutical products; however, other products could be used.

At operation 502, pharmaceutical products are received from a manufacturer or distributor. In one embodiment, the pharmaceutical products may be oral solids. In other embodiments, the pharmaceutical products may comprise encapsulated liquids, gel tablets, injectables, or any other suitable compositions. An inventory of blister cards and RFID films are also received for assembling one or more smart packages.

At operation 504, the pharmaceutical products are placed in each of multiple cells in the blister card. Depending on the type of product and expected use, the products may be placed in the blister card randomly or according to a certain order. For example, when each product is the same (e.g., same medication and same dosage), the products may be placed randomly in the blister card spaces. Alternatively, if the products are different medications or have different dosages, the operator places the products in the cells according to a prescribed medication schedule. Depending on the prescription, one, more than one, or all of the cells in the blister card may be occupied by a product.

At operation 506, a RFID film is placed on the filled blister card so that the cells of the blister card are aligned with RFID tags on the RFID film. The RFID film is formed of paper, plastic, metallic foil, paraffin, any other suitable material, or any combination of these. In a particular embodiment, the RFID film may be a paraffin-coated paper with adhered RFID tags. The blister card is then sealed to the RFID film to create a sealed smart package.

At operation 508, information about each product in the smart package is associated with information about the corresponding RFID tag in one or more digital records. For example, an operator may use a computer application to associate a pre-determined arrangement of RFID tags to a pre-determined medication schedule that corresponds to the products in the smart package. The association may be enabled by the geometry of the smart package. As a particular example, it may be known in the computer application that there are 32 cells in the smart package and the cells are numbered from top-left to bottom-right in a left-to-right, top-to-bottom arrangement. The products may have been placed in each cell in numerical order according to the order that the products should be used. As another example, the operator may use an application to create individual records for each product and its corresponding RFID tag. The records may be stored in one or more memories, such as a database on a network server, a local computer, or a smart chip or other memory on the smart package.

At operation 510, the smart package itself is associated with a patient, or other user, in one or more digital records. For example, the smart package may represent a one-month prescription of a particular medication. The prescription is identified by one or more identifiers, such as a prescription number. Likewise, the smart package itself is associated with a particular identifier, such as a bar code, QR code, serial number, or any other suitable identifier. In some embodiments, the identifier could be a predetermined serial number on the RFID film. For example, a distributor may provide a batch of one hundred RFID films for use in preparing smart packages. Each of the one hundred RFID films may have a unique identifier preprinted on the film. Likewise, identifiers of each of the RFID tags on a particular RFID film may be already associated with the unique identifier of the RFID film. Later, once a smart package has been assembled, the preprinted identifier (e.g., #53 of 100) can be associated with the prescription number, a patient name, a patient number, a customer number, etc., in one or more digital records.

At operation 512, the sealed smart package containing the prescribed pharmaceutical products is distributed to the patient. Once the patient has the smart package, the patient can place the smart package in contact with a smart package reader. As the patient accesses the products from the smart package, information about the used products is read by the smart package reader, and the information can be distributed to one or more vested entities, as described in greater detail below.

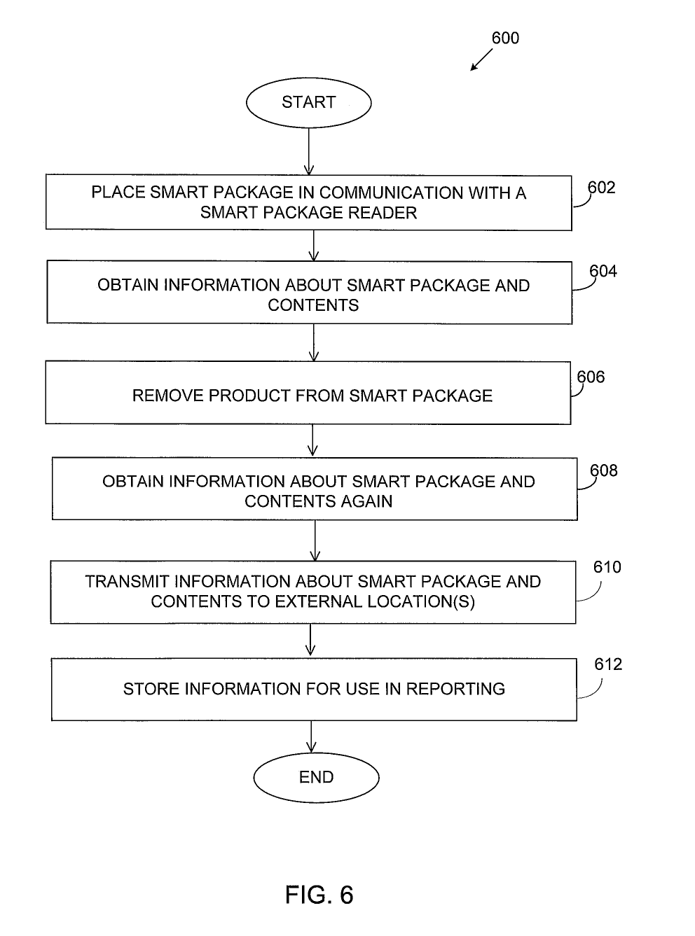

FIG. 6 illustrates an example method for tracking usage of products in a smart package, according to this disclosure. In some embodiments, the method 600 may be performed in conjunction with the product tracking system 100 as described above with reference to FIG. 1. The embodiment of the method 600 illustrated in FIG. 6 is for illustration only. Other embodiments could be used without departing from the scope of this disclosure. The method 600 is described in the context of pharmaceutical products; however, other products could be used.

At operation 602, a prescription of medication packaged in a RFD-enabled smart package is received for a patient (or another user). The smart package is placed where the package is in communication with a smart package reader. For example, in some embodiments, the smart package reader may include one or more slots into which a smart package is placed. The smart package reader can be located in any suitable environment, such as a patient's home, a care provider's office, or any other location where medication may be dispensed.

At operation 604, the smart package reader obtains information about the smart package and its contents. For example, the smart package reader wirelessly monitors each RFID tag associated with the smart package. Additionally, in some embodiments, the smart package reader reads identifier information (e.g., a bar code, a QR code, a smart chip, etc.) associated with the smart package as a whole. The identifier information may include a prescription number, a patient name, a patient number, a customer number, or any other suitable information.

At operation 606, a patient or another user removes the smart package from communication with the reader (e.g., by removing the smart package from the designated slot), dispenses a product from the smart package by pushing the product through a cell cover on the smart package (thereby breaking the associated RFID tag), and returns the smart package to the reader.

At operation 608, when the smart package is returned to the smart package reader, the smart package reader detects that the smart package has been returned and again obtains information about the smart package and its contents, such as by reading each RFID tag associated with the smart package, and optionally reading identifier information associated with the smart package as a whole. This operation may be performed every time the smart package is returned to the smart package reader. In some embodiments, a switch or sensor associated with the slot in the smart package reader may be triggered whenever the smart package is removed from, or returned to, the smart package reader. The trigger of the switch or sensor may act as a trigger for the smart package reader to obtain information about the smart package and its contents.

The smart package reader continues to monitor information about the smart package and its contents by reading the RFID tags that are capable of being read. The reading may be performed at different times, including: continually, regularly according to a predetermined schedule, only when the smart package is removed from and then replaced to the reader, according to a request for updated information, or a combination of two or more of these. The smart package reader may store timestamp information with each read in order to correlate product usage with a particular time of usage.

At operation 610, the information about the smart package and its contents is periodically transmitted from the smart package reader to one or more external locations, such as a network node or a server. Depending on the information available to the smart package reader, the smart package reader may simply transmit identifiers of readable RFID tags associated with a smart package, or may transmit more product-specific information, such as the presence or absence of an individual product. For example, in some embodiments, the smart reader can read product-specific information from an identifier of the smart package, or download product-specific information from an external server, or the smart reader may have product-specific information pre-stored in a memory; thus, the smart reader may be able to correlate a present or absent RFID tag with information about the associated product. In such cases, instead of merely transmitting information that a RFID tag is no longer readable, the smart reader may be able to transmit information that a 50 mg capsule of a particular type of medication has been removed from the smart package (and presumably consumed). Timestamp information obtained during each scan of the RFID tags can also be transmitted, in order to correlate product usage with a particular time of usage.

At operation 612, a server or a network node at a vested entity receives and stores the obtained information about the smart package and its contents. The obtained information may include RFID tag information, associated product information, timestamp information, or any combination of these. Product information may be correlated with timestamp information to generate a usage schedule of a prescribed medication. The usage schedule may then be compared with a recommended usage schedule to confirm that a patient is consuming her medication at the correct time and in the correct dosage. The product information may also be used to determine that a prescription is almost completely consumed and a refill is needed. If multiple smart readers representing multiple patients provide product usage information to a server, such information can be aggregated to determine usage trends for the purposes of maintaining more efficient levels of inventory. For example, a rate of usage may be generated according to a quantity of pharmaceutical product used over a given period of time.

Product usage information may also be correlated with aging, expiration, and recall data. For example, an expired product list may be determined by correlating manufacturer expiration date information with current product inventory data obtained from the server. In such cases, information regarding an expired or recalled product could be transmitted back to the smart reader, where an electronic display may notify a patient not to consume a particular expired or recalled product.

In some embodiments, a server transmits the product information in response to a request for the product information from a network node associated with the vested entity. That is, the product information is transmitted to the vested entity upon a request for such information. In another embodiment, the server periodically transmits the product information to the network node of the vested entity. That is, the server may implement a push-type protocol in which product information is automatically transmitted to certain vested entities at recurring time periods. In either case, access to the product information may be restricted by an authentication process such that only qualified entities may receive such information.

It may be advantageous to set forth definitions of certain words and phrases used throughout this patent document. The term “couple” and its derivatives refer to any direct or indirect communication between two or more elements, whether or not those elements are in physical contact with one another. The terms “include” and “comprise,” as well as derivatives thereof, mean inclusion without limitation. The term “or” is inclusive, meaning and/or. The phrase “associated with,” as well as derivatives thereof, means to include, be included within, interconnect with, contain, be contained within, connect to or with, couple to or with, be communicable with, cooperate with, interleave, juxtapose, be proximate to, be bound to or with, have, have a property of, have a relationship to or with, or the like. The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C.

The various components and operations shown in each of FIGS. 1 through 6 may be incorporated in other figures without departing from the scope of this disclosure. Modifications, additions, or omissions may be made to the systems, apparatuses, and methods described herein without departing from the scope of this disclosure. The components of the systems and apparatuses may be integrated or separated. Moreover, the operations of the systems and apparatuses may be performed by more, fewer, or other components. The methods may include more, fewer, or other operations. Additionally, operations may be performed in any suitable order. As used in this document, “each” refers to each member of a set or each member of a subset of a set.

Various functions described herein can be implemented or supported by one or more computer programs, each of which is formed from computer readable program code and embodied in a computer readable medium. The terms “application” and “program” refer to one or more computer programs, software components, sets of instructions, procedures, functions, objects, classes, instances, related data, or a portion thereof adapted for implementation in a suitable computer readable program code. The phrase “computer readable program code” includes any type of computer code, including source code, object code, and executable code. The phrase “computer readable medium” includes any type of medium capable of being accessed by a computer, such as read only memory (ROM), random access memory (RAM), a hard disk drive, a compact disc (CD), a digital video disc (DVD), or any other type of memory. A “non-transitory” computer readable medium excludes wired, wireless, optical, or other communication links that transport transitory electrical or other signals. A non-transitory computer readable medium includes media where data can be permanently stored and media where data can be stored and later overwritten, such as a rewritable optical disc or an erasable memory device.

While this disclosure has described certain embodiments and generally associated methods, alterations and permutations of these embodiments and methods will be apparent to those skilled in the art. Accordingly, the above description of example embodiments does not define or constrain this disclosure. Other changes, substitutions, and alterations are also possible without departing from the spirit and scope of the disclosure, as defined by the following claims.

What is claimed is:

1. A method for distributing products, comprising:
placing each of a plurality of products into a separate cell of a package, the package having a plurality of cells;
covering the cells of the package with a cover layer having a plurality of radio frequency identification (RFID) tags, such that each RFID tag is aligned with a corresponding cell, and sealing the package; and
associating information about each product with the corresponding RFID tag in one or more first digital records and storing the first digital records in a memory.

2. The method of claim 1, further comprising:
associating identifier information of the package with identifier information of an intended user of the products in one or more second digital records and storing the second digital records in the memory.

3. The method of claim 2, wherein storing the first and second digital records in the memory comprises transmitting the first and second digital records to an external server.

4. The method of claim 1, wherein placing each of the products into the separate cell comprises placing the products in the cells according to an order associated with schedule of intended usage of the products.

5. The method of claim 1, wherein the cover layer comprises a RFID sheet pre-formed with a size and shape substantially the same as the package, the RFID sheet pre-formed with the RFID tags and a unique identifier associated with the RFID sheet.

6. The method of claim 1, wherein the cover layer comprises a paraffin-coated paper.

7. The method of claim 1, wherein the products comprise orally ingested pharmaceuticals.

8. The method of claim 1, wherein the package comprises a blister pack.

9. A method for tracking product usage, the method comprising:
establishing communication with a radio frequency identification (RFID) enabled package, the package having a plurality of cells, each cell containing a product, each cell having a corresponding RFID tag on a surface of the cell;
determining first information associated with the products in the package by wirelessly monitoring each RFID tag;
detecting, after removal of the package from a reader, that the package has been returned to the reader;
determining, after the detection that the package has been returned to the reader, second information associated with the products in the package by wirelessly monitoring each RFID tag again, wherein at least one of the RFID tags is broken and unreadable; and
transmitting the first and second information to a network node associated with at least one vested entity associated with the products,
wherein the first information is different than the second information due to the at least one broken RFID tag.

10. The method of claim 9, wherein the first information and the second information comprise identifiers of each readable RFID tag.

11. The method of claim 9, wherein the first information and the second information further comprise product-specific information of each product associated with each readable RFID tag, wherein a difference between the first information and the second information indicates that a product associated with at least one unreadable RFID tag has been removed from the package.

12. The method of claim 9, further comprising:
reading a package identifier on the package, the package identifier comprising information associated with the package as a whole.

13. The method of claim 12, wherein the products comprise orally ingested pharmaceuticals and the information associated with the package as a whole comprises at least one of a prescription number, a patient name, a patient number, or a customer number.

14. The method of claim 9, wherein the first and second information further comprises timestamp information.

15. An apparatus for tracking product usage, the apparatus comprising:
a radio frequency identification (RFID) reader configured to establish communication with a RFID enabled package, the package having a plurality of cells, each cell containing a product, each cell having a corresponding RFID tag on a surface of the cell;
a data processing system configured to:
determine first information associated with the products in the package by wirelessly monitoring each RFID tag;
determine, after removal of the package from the apparatus, that the package has been returned to the apparatus; and
determine, after the package has been returned to the reader, second information associated with the products in the package by wirelessly monitoring each RFID tag again, wherein at least one of the RFID tags is broken and unreadable; and
a network interface configured to transmit the first and second information to a network node associated with at least one vested entity associated with the products,
wherein the first information is different than the second information due to the at least one broken RFID tag.

16. The apparatus of claim 15, wherein the first information and the second information comprise identifiers of each readable RFID tag.

17. The apparatus of claim 15, wherein the first information and the second information further comprise product-specific information of each product associated with each readable RFID tag, wherein a difference between the first information and the second information indicates that a product associated with at least one unreadable RFID tag has been removed from the package.

18. The apparatus of claim 15, wherein the apparatus is further configured to:
read a package identifier on the package, the package identifier comprising information associated with the package as a whole.

19. The apparatus of claim 18, wherein the products comprise orally ingested pharmaceuticals and the information associated with the package as a whole comprises at least one of a prescription number, a patient name, a patient number, or a customer number.

20. The apparatus of claim 15, wherein the first and second information further comprises timestamp information.
